(19) United States
(12) Patent Application Publication
(10) Pub. No.: US 2014/0274919 A1
(43) Pub. Date: Sep. 18, 2014

(54) BORON-CONTAINING COMPOSITIONS AND METHODS THEREFOR

(71) Applicant: VDF FutureCeuticals, Inc., Momence, IL (US)

(72) Inventor: Zbigniew Pietrzkowski, Aliso Viejo, CA (US)

(73) Assignee: VDF FutureCeuticals, Inc., Momence, IL (US)

(21) Appl. No.: 14/118,865

(22) PCT Filed: May 17, 2012
(86) PCT No.: PCT/US2012/038452

§ 371 Date: May 14, 2014

Related U.S. Application Data
(60) Provisional application No. 61/488,448, filed on May 20, 2011.

(51) Int. Cl. A61K 31/70

(52) U.S. Cl. CPC A61K 31/70
USPC 514/23

(57) ABSTRACT

Contemplated boro-carbohydrate compounds are demonstrated to modulate the blood concentrations of various polypeptides associated with metabolic syndrome by administration of a tetrahedral, and under-carboxylated osteocalcin, adiponectin, and YKL-40. Use of such compounds in oral administration is especially preferred and may be effective to address one or more components of metabolic syndrome.

This application claims priority to our copending U.S. provisional application with the Ser. No. 61/488448, which was filed May 20, 2011.

### FIELD OF THE INVENTION

The field of the invention is compositions and methods for boron-containing compounds and compositions, especially as it relates to nutraceutical and pharmaceutical compositions and uses therefor.

### BACKGROUND OF THE INVENTION

Metabolic syndrome, also known as insulin resistance syndrome, is a collection of disorders that, in combination, greatly increase an individual's chances of developing diabetes and cardiovascular disease. Metabolic syndrome has become a serious health issue in the United States, with up to 25% of the population affected, and is increasingly prevalent as the population ages. In addition, metabolic syndrome is a growing problem in developing countries. Generally speaking, metabolic syndrome is characterized by obesity, difficulty in regulating glucose levels, elevated blood pressure, high serum triglyceride levels, and decreased HDL levels. Difficulties in regulating the body's glucose levels include type II diabetes, which is characterized by a decrease in the body's response to insulin. Biological markers for inflammation, such as C-reactive protein and YLK-40, may be present at elevated levels in affected individuals and are often associated with the development of atherosclerosis. Risk factors for metabolic syndrome include sedentary lifestyle, stress, and age.

At present there is no satisfactory treatment for metabolic syndrome. Lifestyle changes, such as exercise and improved diet, are beneficial for some individuals but often fail to address all of the symptoms. As a result the disorders that characterize metabolic syndrome are often addressed individually and specifically, for example through the use of drugs that reduce serum triglyceride and LDL (so called “bad cholesterol”) levels and the use of drugs that increase the body's response to insulin. This approach may not be satisfactory, however, as it fails to address multiple aspects of the syndrome, for example, inflammation that is associated with atherosclerosis and cardiovascular disease.

Recent findings indicate that certain endogenous peptide hormones have activities that may be beneficial in the treatment of metabolic syndrome. One such hormone is adiponectin, which is produced by adipose tissue. Adiponectin is typically present in the body at relatively high concentrations in healthy individuals, and is reduced in individuals with insulin resistant diabetes, hyperlipidemia, high LDL, coronary artery disease, and obesity (Diez, J., and Iglesias, P. (2003), The Role of the Novel Adipocyte-Derived Adiponectin in Human Disease. European Journal of Endocrinology, 1483: 293-303). Iwashima et al have also noted an association between low adiponectin levels and hypertension, and cite regulation of this hormone as a key component of metabolic syndrome (Iwashima, Y., Katsuya, T., Ishikawa, K., Ouchi, N., Ohishi, M., Sugimoto, K., Fu, Y., Motone, M., Yamamoto, K., Matsuo, A., Ohashi, K., Kihara, S., Funahashi, T., Rakugi, H., Matsuzawa, Y., and Ogihara, T. (2004), Hypoadiponectemia Is an Independent Risk Factor for Hypertension. Hypertension, 41: 20-24). Beyond such associations, adiponectin is also known to have anti-inflammatory and anti-atherosclerotic properties (Renaldi, O., Pramono, B., Sinorita, H., Purnomo, L., Asdie, R., and Asdie, A. (2009), Hypoadiponectemia: A Risk Factor for Metabolic Syndrome. Acta Med Indones-Indones J Intern Med, 43: 1318-1323). Further studies show that administration of adiponectin is effective in inducing weight loss and increasing sensitivity to insulin in animal models (Diez, J., and Iglesias, P. (2003), The Role of the Novel Adipocyte-Derived Adiponectin in Human Disease. European Journal of Endocrinology, 1483: 293-303).

Another peptide hormone that has gathered interest as a potential treatment for metabolic syndrome is osteocalcin, which is produced by osteoblasts. This hormone occurs in multiple forms due to different degrees of post-translational carboxylation (Shea, M., Gundberg, C., Meigs, J., Dallal, G., Saltzman, E., Yoshida, M., Jacques, P., and Booth, S. (2009), Gamma Carboxylation of Osteocalcin and Insulin Resistance in Older Mean and Women. Am J Clin Nutr, 90: 1230-1235). Lee et al found that administration of non-carboxylated osteocalcin to mice increased sensitivity to insulin, perhaps due to regulation of the production of adiponectin (Lee, N., Sowa, H., Hinoi, E., Ferron, M., Ahn, J., Confavreux, C., Dacquin, R., Mee, P., McKee, M., Jung, D., Zhang, Z., Kim, J., Mauvais-Jarvis, F., Ducy, P., and Karsenty, G., P. (2007), Endocrine Regulation of Energy Metabolism by the Skeleton. Cell, 130(3): 456-469). Studies in humans, however, indicate that the relationship between osteocalcin and insulin resistance is more complex in our species. High levels of non-carboxylated osteocalcin are associated with both higher adiponectin levels and increased insulin release in children (Prats-Puig, A., Mas-Parareda, M., Riera-Perez, E., Gonzalez-Forcadell, D., Mier, C., Mallol-Guisset, M., Diaz, M., Bassols, J., Zegher, F., Ibanez, L. and Lopez-Bermejo, A. (2010), Carboxylation of Osteocalcin Affects Its Association with Metabolic Parameters in Healthy Children. Diabetes Care, 33: 661-664), however Shea et al noted a study that shows an association between a decreased percentage of non-carboxylated osteocalcin and protection against the development of insulin resistance in older men.

While appealing in their ability to address multiple aspects of metabolic syndrome, use of such peptide hormones as pharmaceuticals is unfortunately not a simple matter. Peptides are generally expensive to produce, can be unstable at ambient temperatures, and may require post-translational modification to be effective. Since peptides are often rapidly degraded in the digestive tract, peptide drugs are in many cases administered by injection, which is both inconvenient and uncomfortable. In some instances, such as adiponectin, the peptide hormone may already be present in the body at high enough concentrations that doses sufficient to produce a significant change are impractical. Some investigators have therefore attempted to utilize compounds that modulate the body's production of such hormones. W02006/034435A2, for example, discloses glutamic acid boroproline compounds that are reported to increase adiponectin levels to at least some degree. Such compounds, however, require a complex multistep synthetic process and may be susceptible to oxidation.

Thus, there remains a growing need for inexpensive and conveniently administered compounds that are effective in modulating endogenous substances related to metabolic syndrome.

### SUMMARY OF THE INVENTION

The inventive subject matter provides methods and kits comprising a tetrahedral boro-carbohydrate complex that can modulate concentrations of endogenous substances related to metabolic syndrome. The boro-carbohydrate complex may include one or more carbohydrate molecules, and the carbohydrate is preferably a cis-diol capable of forming stable complexes with borate. While in some embodiments of the inventive subject matter the carbohydrate is fructose, in other embodiments of the inventive subject matter the borate complex includes a cation, for example, a calcium ion.

In especially preferred aspects of the inventive subject matter, a method is presented that modulates the concentration and/or activity of an endogenous substance that is associated with metabolic syndrome (or related conditions) by administration of a boro-carbohydrate complex to a mammal, typically at an effective dosage and schedule. Preferred endogenous substances include osteocalcin, adiponectin, and/or YKL-40. For example, contemplated methods may modulate under-carboxylated form of osteocalcin, which also include non-carboxylated osteocalcin. Inflammatory markers that may be modulated by the inventive subject matter include YLK-40. In further embodiments, concentrations of adiponectin may be increased, while under-carboxylated osteocalcin and/or YKL-40, may be decreased. An effective dose of the boro-carbohydrate complex may be 0.01 mg or more per administration, which is most preferably oral.

Another embodiment of the inventive subject matter, a kit is contemplated that has a boro-carbohydrate complex in an amount effective to modulate the concentration of one or more endogenous substances related to metabolic syndrome and directions for use. Such a kit may include a container that encloses a boro-carbohydrate complex in a convenient dosage form such as, for example, a pill, a capsule, an oral suspension or solution, or an injectable suspension or solution. In some embodiments of the inventive subject matter, the directions for use describe utilization of the contents of the kit for treatment of metabolic syndrome. In another embodiment of the inventive subject matter, the directions for use describe utilization of the contents of the kit for treatment of disorders associated with metabolic syndrome. Such disorders include obesity, insulin resistance, elevated blood glucose concentrations, elevated blood pressure, high serum triglyceride concentration, decreased high density lipoprotein concentration, increased low density lipoprotein concentration, atherosclerosis, cardiovascular disease, and/or inflammation. In still other embodiments of the inventive subject matter, the directions for use describe utilization of the contents of the kit for modulation of the concentration of an endogenous substance associated with metabolic syndrome. Such endogenous substances especially include YKL-40, under-carboxylated osteocalcin (including non-carboxylated osteocalcin) and/or adiponectin.

Various objects, features, aspects and advantages of the inventive subject matter will become more apparent from the following detailed description of preferred embodiments, along with the accompanying drawing figures.

### BRIEF DESCRIPTION OF THE DRAWINGS

FIG. 1 shows an exemplary effect of oral administration of a calcium fructoborate complex on the concentration of undercarboxylated osteocalcin in blood.

FIG. 2 shows an exemplary effect of oral administration of a calcium fructoborate complex on the concentration of adiponectin in blood.

FIG. 3 shows an exemplary effect of oral administration of a calcium fructoborate complex on the concentration of YKL-40 in blood.

FIG. 4 shows an exemplary effect of oral administration of a calcium fructoborate complex on the concentration of C-reactive protein in blood.

### DETAILED DESCRIPTION

The inventors have surprisingly discovered that administration of a tetravalent borate compound can have the previously unknown effect of modulating the concentrations of certain proteins associated with metabolic syndrome. Such proteins especially include under-carboxylated osteocalcin, adiponectin, and YKL-40. In this context it should be noted that non-carboxylated (alternatively referred to as uncarboxylated osteocalcin) is considered a form of under-carboxylated osteocalcin.

Numerous tetravalent boron-containing compounds, including boron-containing carbohydrate complexes and certain uses thereof are well known in the art and are described, for example, in U.S. Pat. Nos. 5,962,049, 5,985,842, 6,080,425, 6,696,419, and 6,924,269, and U.S. Pat. App. Nos. 2005/0032743 and 2010/0256076. These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply. Among other effects, such compounds were previously reported to increase steroid homeostasis, act as anti-inflammatory agents, and to improve dietary deficiency of nutritionally available boron. Advantageously, almost all of these boron-containing compounds are relatively simple to prepare, non-toxic in even relatively large quantities, and are effective and typically well tolerated upon simple oral administration.

In some embodiments of the inventive subject matter the tetravalent borate compound is a boro-carbohydrate complex, formed by the well known interaction between boron and cis-diol containing compounds. Exemplary cis-diol containing compounds include various sugars, sugar alcohols, and polyols. Especially preferred sugars that form such complexes include fructose, mannose, xylose, and sorbose. Boro-carbohydrate complexes may also be charged, and such charged complexes may be complexed with cations in order to provide neutralization. Cations that are useful for this purpose include sodium, potassium, calcium, and magnesium. In a particularly preferred aspect of the inventive subject matter, the boro-carbohydrate complex is a fructoborate complex that is charge-neutralized with a calcium ion to form a calcium fructoborate complex (CFB).

FIG. 1 shows the results of an exemplary trial of oral administration of CFB to human volunteers. In this instance the effect of administration of a CFB on the concentration of under-carboxylated osteocalcin in the blood of human volunteers, along with the effect of administration of sodium borate (Na borate) and a placebo compound was measured. Blood samples were taken at day 0 prior to administration in order to establish a baseline concentration for each individual. Additional samples were taken after 7 and 14 days of treatment. Under-carboxylated osteocalcin was characterized using a commercially available immunoassay from Cusabio Biotech Co. LTD (Newark, Del.) according to the manufacturer's directions, and the percentage change from pretreatment concentrations of under-carboxylated osteocalcin were calculated. While there was no discernable effect on total osteocalcin (data not shown), a significant reduction in the concentration of under-carboxylated osteocalcin is evident in the group treated with CFB. In some embodiments of the inventive subject matter the percentage of under-carboxylated osteocalcin (including non-carboxylated osteocalcin) may thus be reduced by about 1%, 2%, 3%, 4%, 5%, 10%, 15%, 20%, 25%, 30%, 35%, 40%, 45%, 50% or more relative to pretreatment values following oral administration of CFB.

While studies have indicated a connection between under-carboxylated osteocalcin and aspects of metabolic syndrome, modulation of under-carboxylated osteocalcin may have additional or alternative effects in other body functions where this hormone is known to be involved. The inventor therefore also contemplates that various components and parameters of the human body, particularly those related to the skeletal system and the deposition and/or activity of adipose tissue, may be modified by oral administration of CFB (or other boro-carbohydrate complexes discussed herein), particularly in an elderly female population that shows significant postmenopausal changes in blood concentrations of uncarboxylated osteocalcin. It is also contemplated that such boro-carbohydrate complexes may have utility in regulating the number and activity of mitochondria, in fat storage and metabolism, in glucose utilization, and in the accumulation of hepatic fat. In addition, since species of osteocalcin may influence the production or otherwise regulate the activity of other molecules that mediate processes in skeletal and adipose tissues, the inventor contemplates that the boro-carbohydrate complexes presented herein may also be utilized to modulate activities influenced by mediators such as leptin, leptin receptors, insulin, insulin receptors, FGF-21, and adiponectin.

As noted above, osteocalcin occurs in carboxylated and under-carboxylated forms, which is thought to be a result of post-translational modification of the peptide. As it was found that the concentration under-carboxylated osteocalcin was decreased in subjects treated with a boro-carbohydrate complex without affecting the total osteocalcin concentration (data not shown), the inventor contemplates that this change in distribution between carboxylated and under-carboxylated forms of osteocalcin may be due to modulation in the activity of the carboxylation process. It should therefore be appreciated that administration of a boro-carbohydrate complex may increase the activity of one or more carboxylases (particularly gamma-carboxylase). This may occur through various mechanisms, including increased enzyme production, increased enzyme release, and decreased activity of enzyme inhibitors. Alternatively, a boro-carbohydrate complex may act to release or otherwise increase the availability of enzyme cofactors, such as vitamin K. As a consequence, it is contemplated that boro-carbohydrate complexes may also be useful in the modulation of biomolecules that are dependent on carboxylation processes, and may have utility in treatment of conditions associated with such biomolecules. For example, the boro-carbohydrate complexes of the inventive subject matter are deemed to be useful for modification of blood clotting and dentin disorders.

FIG. 2 shows the results of another exemplary trial of oral administration of CFB to human volunteers. In this instance the effect of oral administration of CFB on the blood concentration of adiponectin, along with the effect of administration of sodium borate (Na borate), and a placebo compound was measured. Blood samples were taken at a time 0 prior to administration in order to establish a baseline concentration for each individual. Additional samples were taken at 30 minute intervals, and the concentration of adiponectin compared to the baseline level determined for each individual. It is readily apparent that administration of a boro-carbohydrate complex had a profound effect on a hormone associated with metabolic syndrome, inducing a rapid and sustained increase in the concentration of adiponectin. Surprisingly, sodium borate was found to have a similar, but markedly reduced, effect. The mechanism for this increase in adiponectin concentration in the blood is not known, but it is contemplated that it may due to increased synthesis, increased release of stored adiponectin, or a combination of these. Therefore, in some embodiments of the inventive subject matter blood concentrations of adiponectin may be increased by about 1%, 2%, 3%, 4%, 5%, 10%, 15%, 20%, 25%, 30%, or more relative to pretreatment values following administration of a boro-carbohydrate complex.

In all of the above oral administrations, the human volunteers were provided with orally administered single daily doses of 5-50 mg of calcium fructoborate for at least 14 days. However, it should be noted that the orally administered compositions may vary considerably, so long as at least 0.1 mg, more typically at least 1.0 mg, and most typically at least 10-100 mg of the compound are administered to the human (or other mammal). Thus, typical administrations will provide 0.001-0.01 mg/kg, more typically 0.01-0.1 mg/kg, and most typically 0.1-5.0 mg/kg (and in some cases even higher) of the boro-carbohydrate compound per day. Such administration may be performed in numerous formats, and all types of formulations comprising contemplated compounds are deemed suitable for use herein, including as nutritional supplement, as ingredient in a snack (e.g., energy bar, fruit leather, etc.), a cereal, a beverage, etc.

It should, therefore, be appreciated that the administration, and particularly oral administration of compounds and compositions presented herein may modify, reduce, or even reverse undesirable conditions that are associated with reduced or low blood concentrations of adiponectin. Adiponectin has been shown to have an impact on a variety of conditions associated with metabolic disorder (Matsuzawa, Y., Funahashi, T., Kihara, S. and Shimomura, I. (2003), Adiponectin and Metabolic Syndrome. Arterioscler Thromb Vasc Biol 24: 29-33), particularly obesity, cardiovascular disease, impaired glucose uptake and/or gluconeogenesis, impaired or reduced insulin release, decreased insulin sensitivity, hyperlipidemia, hypertension, and vascular changes that can lead to atherosclerosis. It is therefore contemplated that administration of a boro-carbohydrate complex may be effective in modifying such disorders.

In still further contemplated aspects of the inventive subject matter it is contemplated that various aspects of ageing, and especially metabolic slow-down and bone-degradation could be modified, slowed, or even reversed using the compounds and compositions contemplated herein. For example, it is known that adiponectin plays a role in the suppression of various metabolic derangements associated with aging that may result in type 2 diabetes, obesity, atherosclerosis, and non-alcoholic fatty liver disease.

While studies have indicated a connection between adiponectin and aspects of metabolic syndrome, modulation of adiponectin may have additional or alternative effects in other body functions where this hormone is known to be involved. For example, adiponectin is known to be a negative regulator of the angiogenesis that is an important component in the formation of many tumors. It is therefore contemplated that administration of a boro-carbohydrate complex may be effective in reducing angiogenesis and in modifying disorders associated with angiogenesis activity.

FIG. 3 shows the results of yet another exemplary trial of oral administration of CFB. In this instance the effect of the administration of CFB on the concentration of YKL-40 in the blood of human volunteers, along with the effect of administration of sodium borate (Na borate) and a placebo compound, was measured. Blood samples were taken at Day 0 prior to administration in order to establish a baseline concentration for each individual.

Additional samples were taken after 7 and 14 days of treatment, and the concentration of YKL-40 compared to the baseline level determined for each individual. YKL-40 was characterized using a commercially available immunoassay from Quidel (San Diego, Calif.) according to the manufacturer's directions, and the percentage change from pretreatment concentrations of YKL-40 were calculated. A significant reduction in the concentration of YKL-40 is evident in the group treated with CFB. Therefore, in some embodiments of the inventive subject matter the percentage of YKL-40 may be reduced by 1%, 2%, 3%, 4%, 5%, 10%, 15%, 20%, 25%, 30%, 35%, 40%, or more relative to pretreatment values following administration of a boro-carbohydrate complex.

It should be appreciated that blood concentrations of YKL-40 are often elevated in a number of conditions associated with metabolic syndrome, and may be used as a “marker” for the presence or characterization of the severity of these. For example, YKL-40 is elevated in obesity, insulin-resistant diabetes, cardiovascular disease, atherosclerosis, and hypertension. While this does not necessarily indicate a causal relationship between YKL-40 and such conditions the inventor contemplates that such an association may indicate that elevated YKL-40 and disorders associated with metabolic syndrome may have a common underlying origin. The inventor further contemplates that administration of a boro-carbohydrate complex may therefore have utility in modifying such disorders in addition to reducing YKL-40 concentrations in blood.

While often used as a diagnostic marker, YKL-40 has known biological activities associated with a variety of disease conditions. For example, YKL-40 is a promoter of angiogenesis associated with the formation of many tumors. It is therefore contemplated that administration of a boro-carbohydrate complex may be effective in reducing angiogenesis and in modifying conditions associated with angiogenesis activity. YKL-40 has also been implicated in tissue remodeling associated with rheumatoid arthritis, osteoarthritis, and other inflammatory joint diseases. Boro-carbohydrate complexes, therefore, may have utility in reducing tissue remodeling and in mitigating the effects of such conditions.

Additionally, it is contemplated that compounds and compositions according to the inventive subject matter may influence various parameters associated with an inflammatory response, as YKL-40 has been proposed as a diagnostic marker for inflammation. Most significantly, the inventor noted that oral administration of CFB, in addition to significantly reducing the blood concentration of YKL-40 (as noted above and as seen in FIG. 3) also reduced blood concentrations of a second inflammation marker, C-reactive protein (CRP), an unrelated protein that is synthesized in the liver in response to acute and chronic inflammation. This can be readily taken from FIG. 4, which shows the effect of the oral administration of CFB, sodium borate (Na borate), and a placebo on the concentration of circulating CRP. It is readily apparent that CFB has a substantially greater effect on reducing the concentration of CRP than sodium borate. Thus, it should be appreciated that contemplated compositions are especially advantageous in reduction of inflammatory markers and conditions associated with increased levels of such markers.

It is notable that while administration of boro-carbohydrate complexes have impacted osteocalcin, adiponectin, and YKL-40, these polypeptides are expressed in different tissues. Osteocalcin is expressed by osteocytes found in skeletal tissue. Adiponectin is expressed in adipocytes found in fatty tissue. YKL-40 is expressed in chondrocytes found in cartilaginous tissue and in macrophages and neutrophils that are part of the immune system. All of these tissues, however, have a common developmental lineage, being derived from mesodermal cells. In yet another aspect of the inventive subject matter, therefore, the inventor also contemplates that boron-containing compounds and compositions may be employed as a direct or indirect modulator of stem cell development, and with that as a modulator of adipose tissue generation, immune system activity, and/or functionality and turnover of bone and joint tissue. Thus, contemplated compositions may be useful for management of adult stem cells to maintain functionality of bone/joint tissue (e.g., osteoblasts, fibroblasts, chondrocytes), adipose tissue (e.g., peripheral, omental, visceral), and the immune system (e.g. macrophages, neutrophils, eosinophils, lymphocytes).

Based on the present results and other factors, the inventor also contemplates that an elevated blood concentration of under-carboxylated osteocalcin is reflective of compromised bone health and function, and that such elevated concentrations may force metabolism into an accelerated mode (e.g., via heightened insulin sensitivity, pancreatic activity, mitochondrial activity and number, beta oxidation, etc.) regardless of the actual energy metabolism status. Thus, elevated blood levels of under-carboxylated osteocalcin may function as a metabolic activator. Conversely, clinically normal or reduced blood concentrations of under-carboxylated osteocalcin may be indicative of normal bone health and function, and as a result energy metabolism that is regulated in a caloric intake-dependent manner. Contemplated boron-containing compounds may interfere with the process of carboxylation, either directly by stimulating carboxylase activity, or indirectly by modulating expression of the Esp gene (Hinoi, E., Gao, N., Jung, D. Y., Yadav, V., Yoshizawa, T., Kajimura, D., Myers, Jr., M. G., Chua, Jr., S. C., Wang, Q., Kim, J. K., Kaestner, K. H. and Karsenty, G. (2009), An Osteoblast-dependent Mechanism Contributes to the Leptin Regulation of Insulin Secretion. Annals of the New York Academy of Sciences, 1173: E20-E30).

In yet further contemplated aspects of the inventive concept, the boro-carbohydrate compounds are administered as a single active component. However, in other aspects the boro-carbohydrate compounds may be administered in conjunction with one or more agents effective in modifying a biological activity. Particularly contemplated agents include vitamin K and derivatives thereof, vitamin D and variants thereof (e.g., calcitriol), chromium (and especially chromium compositions as described in U.S. Pat. App. No. 2006/0029642), and other metals in various forms, including copper, magnesium, calcium, etc. Such combination products are expected to have an at least additive, and in some cases even synergistic effect with respect to the biological action. Among other actions, it is assumed that additive or synergistic effect is found with at least one of glucose uptake, fat reduction, increase in beta oxidation, normalization of dyslipidemia, and increase of bone mass and/or density specific embodiments, compositions, and methods related to boron-containing compounds.

Most typically, the dosage range of the boro-carbohydrate complexes (or other boron-containing compound) will be such that administration of boron is between about 0.01 mg and about 100 mg per day, and more typically between about 0.1 and about 15 mg per day. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary. This may administered as a single dose or as a series of doses administered throughout the day. Similarly, treatment duration may be between single administration and administration over several days, weeks, or even months. However, other dosage ranges and administration schedules are also deemed suitable for use herein, and the desired effect will at least to some degree depend on the particular dosage and dosing regime. The boro-carbohydrate complex may be administered orally, parenterally, intravenously, or as a drop administered to the eye or ear canal. In a preferred embodiment the boro-carbohydrate complex or other boron containing compound is administered orally.

A further aspect of the inventive subject matter is drawn to a kit for packaging and/or distributing a boro-carbohydrate complex and instructions or directions for use of such a compound in modifying a condition that is associated with metabolic syndrome. Conditions associated with metabolic syndrome include impaired glucose regulation, hyperglycemia, obesity, impaired insulin release, reduced insulin sensitivity, elevated glycosylated hemoglobin, hypertension, atherosclerosis, cardiovascular disease, inflammation, elevated under-carboxylated osteocalcin, hypoadiponectinemia, and elevated YKL-40. Such a kit may contain a container or enclosure for a boro-carbohydrate complex or other boron containing compound, which may be provided as a solid or a liquid. The boro-carbohydrate complex or other boron containing compound may be supplied in a unit dose form or as a bulk preparation from which unit doses are measured out and administered. Suitable containers include, but are not limited to, bottles, bags, boxes, vials, blister packs, ampoules, dropper bottles, and syringes. Directions may include information related to dosage and schedule of administration. In some embodiments the directions for use may form part of or be affixed to the enclosure for the boro-carbohydrate complex or other boron containing compound. In other embodiments the directions include information related to reduction in concentrations of under-carboxylated osteocalcin, non-carboxylated osteocalcin, and/or YKL-40. In other embodiments the directions may include information related to increasing the concentration of adiponectin. In still other embodiments the kit may include a boro-carbohydrate complex or other boron containing compound and a second compound effective in modifying a biological function.

Thus, specific embodiments, compositions, and methods related to boron-containing compounds and compositions containing such compounds have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, where a definition or use of a term in an incorporated reference is inconsistent or contrary to the definition of that term provided herein, the definition of that term provided herein applies and the definition of that term in the reference does not apply.

What is claimed is:

1. A method of reducing the concentration of under-carboxylated osteocalcin in the blood of a mammal, comprising administering a boro-carbohydrate complex at a dosage and schedule effective to reduce the concentration of under-carboxylated osteocalcin in blood.

2. The method of claim 1, wherein the boro-carbohydrate complex is calcium fructoborate.

3. The method of claim 1, wherein the boro-carbohydrate complex is administered orally.

4. The method of claim 1, wherein the dosage of the boro-carbohydrate complex is at least 10 mg.

5. The method of claim 1, wherein the concentration of under-carboxylated osteocalcin in blood is reduced by at least 10%.

6. The method of claim 1 wherein administration of the boro-carbohydrate complex at the dosage and schedule also increases a concentration of adiponectin in the blood of the mammal.

7. The method of claim 1 wherein administration of the boro-carbohydrate complex at the dosage and schedule also reduces a concentration of YKL-40 in the blood of the mammal.

8. A method of increasing the concentration of adiponectin in the blood of a mammal, comprising administering a boron-carbohydrate complex at a dosage and schedule effective to increase the concentration of adiponectin in blood.

9. The method of claim 8 wherein the boro-carbohydrate complex is calcium fructoborate.

10. The method of claim 8, wherein the boro-carbohydrate complex is administered orally.

11. The method of claim 8, wherein the dosage of the boro-carbohydrate complex is at least 10 mg.

12. The method of claim 8, wherein the concentration of adiponectin in blood is increased by at least 5%.

13. The method of claim 8 wherein administration of the boro-carbohydrate complex at the dosage and schedule also reduces a concentration of under-carboxylated osteocalcin in the blood of the mammal

14. The method of claim 8 wherein administration of the boro-carbohydrate complex at the dosage and schedule also reduces a concentration of YKL-40 in the blood of the mammal.

15. A method of reducing the concentration of YKL-40 in the blood of a mammal, comprising administering a boro-carbohydrate complex at a dosage and schedule effective to reduce the concentration of YKL-40 in blood.

16. The method of claim 15, wherein the boro-carbohydrate complex is calcium fructoborate.

17. The method of claim 15, wherein the boro-carbohydrate complex is administered orally.

18. The method of claim 15, wherein the dosage of the boro-carbohydrate complex is at least 0.01 mg.

19. The method of claim 15, wherein the concentration of YKL-40 in blood is decreased by at least 5%.

20. The method of claim 15 wherein administration of the boro-carbohydrate complex at the dosage and schedule also reduces a concentration of under-carboxylated osteocalcin in the blood of the mammal or increases a concentration of adiponectin in the blood of the mammal.
